(19) United States
(12) Patent Application Publication
(10) Pub. No.: US 2026/0317505 A1
(43) Pub. Date: Oct. 15, 2026

(54) PHOTOTHERMAL RECYCLING OF POLYSTYRENE TO STYRENE MONOMER

(71) Applicants: The Trustees of Princeton University, Princeton, NJ (US); Cornell University, Ithaca, NY (US)

(72) Inventors: Erin E. STACHE, Princeton, NJ (US); Sewon OH, Ithaca, NY (US); Hanning JIANG, Princeton, NJ (US)

(73) Assignees: Cornell University, Ithaca, NY (US); The Trustees of Princeton University, Princeton, NJ (US)

(21) Appl. No.: 19/097,160

(22) Filed: Apr. 1, 2025

Related U.S. Application Data
(60) Provisional application No. 63/572,434, filed on Apr. 1, 2024.

(51) Int. Cl. C08J 11/16; C07C 15/46; C08J 3/28

(52) U.S. Cl. CPC C08J 11/16; C07C 15/46; C08J 3/28; C08J 2325/10; C08J 2325/12; C08J 2325/14

(57) ABSTRACT

The present disclosure provides a method for recycling polystyrene. The method includes providing a photothermal colorant and a polystyrene, with the photothermal colorant being operably coupled to the polystyrene. The method further includes causing the photothermal colorant to generate heat by irradiating the photothermal colorant with light. A styrene monomer is formed by allowing the heat to degrade the polystyrene. The disclosure also provides a system for polystyrene recycling, including a first vessel configured to receive the photothermal colorant and polystyrene, a light source configured to irradiate the photothermal colorant within the first vessel, a vacuum source configured to be operably coupled to the first vessel, and a second vessel configured to receive the styrene monomer from the first vessel.

### CROSS-REFERENCE TO RELATED APPLICATIONS

This application claims priority to U.S. Application No. 63/572,434, titled Recycling Commercial Black Polystyrene Plastic to Monomer Using Photothermal Conversion, filed Apr. 1, 2024, which is hereby incorporated by reference in its entirety.

### STATEMENT REGARDING FEDERALLY SPONSORED RESEARCH OR DEVELOPMENT

This invention was made with government support under Grant No. DE-SC0024412 awarded by the Department of Energy. The government has certain rights in the invention.

### FIELD OF INVENTION

The present disclosure relates to recycling methods and systems for plastic materials, and more particularly to a method and system for recycling polystyrene using photothermal conversion.

### BACKGROUND

Plastics have become an integral part of modern society due to their versatility, durability, and low cost. However, the widespread use of plastics has led to significant environmental challenges, particularly in waste management and recycling. Among various types of plastics, polystyrene is widely used in packaging, disposable consumer goods, and construction materials.

Recycling polystyrene presents several difficulties. Traditional mechanical recycling methods often result in degraded material properties, limiting the applications of recycled polystyrene. Chemical recycling approaches, such as pyrolysis, can break down polystyrene into its constituent monomers, but these processes typically require high energy inputs and specialized equipment.

Black polystyrene products pose additional recycling challenges. The carbon black pigments used to color these plastics interfere with optical sorting systems commonly used in recycling facilities. As a result, black polystyrene items are frequently diverted to landfills or incineration, even in areas with established plastic recycling programs.

There is growing interest in developing more efficient and sustainable methods for recycling polystyrene, particularly for colored variants. Ideally, such methods would be able to process polystyrene waste with minimal energy input and produce high-quality recycled materials or chemical feedstocks.

Photothermal conversion has emerged as a promising approach in various fields, including materials processing and chemical synthesis. This technique involves the use of light-absorbing materials to generate localized heat, potentially offering a way to initiate chemical reactions or material transformations with reduced overall energy consumption.

Advances in recycling technologies that can effectively process black polystyrene and other challenging plastic waste streams could contribute to improved resource efficiency and reduced environmental impact of plastic materials. Such developments may help address broader concerns about plastic pollution and support the transition towards a more circular economy for plastics.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for recycling polystyrene is provided. The method includes providing a photothermal colorant and a polystyrene, the photothermal colorant being operably coupled to the polystyrene. The method further includes causing the photothermal colorant to generate heat by irradiating the photothermal colorant with light. The method also includes forming a styrene monomer by allowing the heat to degrade the polystyrene.

According to other aspects of the present disclosure, the method may include one or more of the following features. The photothermal colorant may be carbon black. The photothermal colorant may be a material other than carbon black. The photothermal colorant and the polystyrene may be separately added to a vessel. The polystyrene may be a polystyrene copolymer. The polystyrene copolymer may be selected from the group consisting of a styrene-acrylic copolymer, a styrene-butadiene copolymer, a styrene-acrylonitrile copolymer, a styrene-acrylonitrile-butadiene copolymer, and a styrene-isoprene copolymer. The photothermal colorant and the polystyrene may be disposed within one or more products configured to be recycled. The styrene monomer may be formed in a vessel under a dynamic vacuum. The styrene monomer may be distilled to a receiving vessel. The photothermal colorant may generate sufficient heat to expose the polystyrene to a temperature of at least about 395 °C. Irradiating the photothermal colorant with light may comprise exposing the photothermal colorant to radiation emitted from an LED. Irradiating the photothermal colorant with light may comprise exposing the photothermal colorant to radiation emitted from the sun. The method may further comprise directing sunlight to the photothermal colorant using a Fresnel lens. Irradiating the photothermal colorant with light may occur for a period of time of at least 5 minutes. The period of time may be 15-60 minutes. The period of time may be at least 60 minutes.

According to another aspect of the present disclosure, a system for polystyrene recycling is provided. The system includes a first vessel configured to receive a photothermal colorant and a polystyrene. The system also includes a light source configured to irradiate the photothermal colorant within the first vessel. The system further includes a vacuum source configured to be operably coupled to the first vessel. The system also includes a second vessel configured to receive a styrene monomer from the first vessel.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a system diagram of a polystyrene recycling apparatus, according to aspects of the present disclosure.

FIG. 2 illustrates a flowchart of a method for recycling polystyrene, in accordance with example embodiments.

FIG. 3 is a schematic and table showing a process for synthesizing black polystyrene via photothermal emulsion polymerization and depolymerized photothermally.

FIG. 4 is a schematic showing a process for PS-co-PMA-CB_{5.3}, PS-co-PAN-CB_{5.3}, and PS-co-PI-CB_{5.1} synthesized via photothermal emulsion polymerization and depolymerized photothermally.

FIG. 5 is a schematic showing a process for HIPS-CB_{6.2} synthesized via photothermal bulk polymerization and depolymerized photothermally

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to methods and systems for recycling polystyrene. In some cases, the methods may involve using photothermal colorants to facilitate the degradation of polystyrene into styrene monomer.

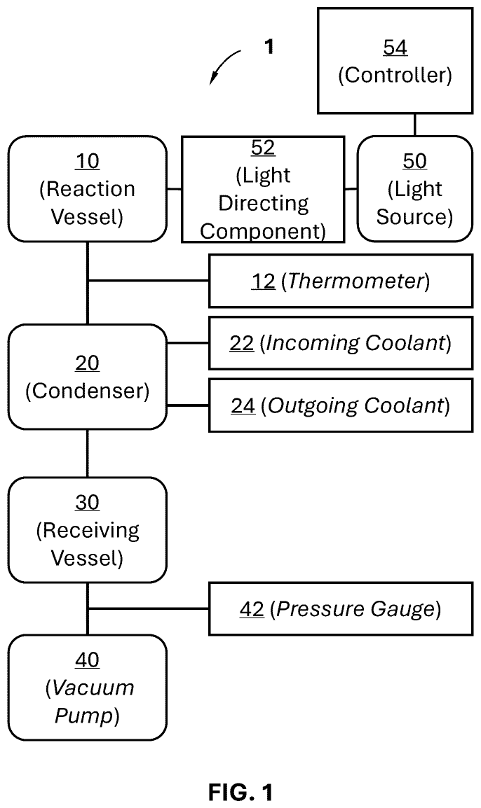

A block diagram of an embodiment of a recycling system is shown in FIG. 1. The recycling system 1 includes several interconnected components. A reaction vessel 10 may be used to receive polystyrene and a photothermal colorant.

In some cases, the photothermal colorant and the polystyrene may be separately added to the reaction vessel 10. This separate addition may allow for precise control over the ratio of photothermal colorant to polystyrene and may facilitate uniform distribution of the colorant within the polystyrene matrix.

### Reaction Vessel

The reaction vessel 10 may be made of materials capable of withstanding the temperatures generated during the recycling process. In some cases, the reaction vessel 10 may be constructed from heat-resistant glass, such as borosilicate glass, which may allow for visual monitoring of the degradation process. Alternatively, the reaction vessel 10 may be made of metals such as stainless steel or aluminum, which may provide excellent heat conductivity and durability.

The shape and size of the reaction vessel 10 may vary depending on the scale of the recycling operation. In some cases, the reaction vessel 10 may be cylindrical to promote uniform heat distribution. The reaction vessel 10 may have a wide mouth to facilitate easy loading of polystyrene and photothermal colorant, as well as cleaning between batches. In other cases, the reaction vessel 10 may have a more complex geometry designed to optimize light absorption and heat transfer.

The reaction vessel 10 may be designed to allow the photothermal colorant to generate sufficient heat to expose the polystyrene to a temperature of at least about 395 °C. This temperature may be necessary to initiate and sustain the depolymerization of polystyrene into styrene monomer. The ability of the reaction vessel 10 to withstand and maintain these high temperatures may be crucial for efficient recycling.

In some implementations, the reaction vessel 10 may include internal baffles or agitators to promote mixing and uniform heat distribution throughout the polystyrene material. These features may help ensure that all parts of the polystyrene sample are exposed to the necessary temperatures for depolymerization.

The reaction vessel 10 may be designed with considerations for thermal insulation and heat management. In some cases, the vessel may have a double-walled construction with a vacuum or insulating material between the walls to minimize heat loss to the environment. This design may help maintain the high temperatures needed for polystyrene degradation while improving energy efficiency.

The reaction vessel 10 may be equipped with ports or connections for attaching additional components. For example, the vessel may have a port for connecting to a vacuum source, allowing for the creation of a reduced pressure environment that may facilitate the collection of styrene monomer. The reaction vessel 10 may also have ports for introducing inert gases or for sampling during the recycling process. The reaction vessel 10 may have an opening for introducing the photothermal colorant and polystyrene, and may be sealable to maintain a vacuum environment.

In some cases, the reaction vessel 10 may be designed with a transparent or translucent section to allow the light from the light source 50 to penetrate and reach the photothermal colorant. This design may be particularly important when using the light directing component 52 to focus or concentrate light onto specific areas within the reaction vessel 10.

### Polystyrene

The polystyrene material used in the recycling process may encompass a wide range of polystyrene types and forms. In some cases, the polystyrene may be pure polystyrene homopolymer. Pure polystyrene may be derived from various sources, including consumer products, industrial waste, or virgin polystyrene materials. In some cases, the polystyrene may be recovered polystyrene from a previous attempt at recycling.

In some cases, the polystyrene may be a polystyrene copolymer. Polystyrene copolymers may incorporate additional monomers alongside styrene to modify the properties of the resulting polymer. The polystyrene copolymer may be selected from the group consisting of a styrene-acrylic copolymer, a styrene-butadiene copolymer, a styrene-acrylonitrile copolymer, a styrene-acrylonitrile-butadiene copolymer, and a styrene-isoprene copolymer.

Styrene-acrylic copolymers may combine styrene with acrylic monomers such as methyl methacrylate or butyl acrylate. These copolymers may offer improved weatherability and chemical resistance compared to pure polystyrene.

Styrene-butadiene copolymers may incorporate butadiene monomers, which may enhance the impact strength and flexibility of the material. These copolymers may be used in applications requiring improved toughness.

Styrene-acrylonitrile copolymers may combine styrene with acrylonitrile, potentially resulting in a polymer with enhanced chemical resistance and thermal stability compared to pure polystyrene.

Styrene-acrylonitrile-butadiene copolymers, also known as ABS (Acrylonitrile Butadiene Styrene), may incorporate all three monomers to create a material with a balance of strength, rigidity, and chemical resistance.

Styrene-isoprene copolymers may combine styrene with isoprene monomers, potentially resulting in materials with elastomeric properties.

The polystyrene material may be present in various forms when subjected to the recycling process. In some cases, the polystyrene may be in the form of consumer products such as food packaging, disposable cutlery, or electronic device housings. The presence of additives, contaminants, or mixed materials in consumer products may affect the recycling process and may require additional preprocessing steps.

In some cases, the polystyrene may be in the form of industrial waste, such as off-cuts, rejected parts, or post-industrial scrap. Industrial waste polystyrene may have a more consistent composition compared to consumer products, potentially simplifying the recycling process.

The physical form of the polystyrene may vary and may affect the recycling process. In some cases, the polystyrene may be in the form of solid objects, sheets, or films. In other cases, the polystyrene may be in the form of foam or expanded polystyrene (EPS). The physical form may influence factors such as heat transfer efficiency and the rate of depolymerization during the recycling process.

In some implementations, the photothermal colorant and polystyrene may be part of existing products to be recycled. For example, black or dark-colored polystyrene products may already contain carbon black or other photothermal colorants as pigments. These products may be directly subjected to the recycling process without the need for additional photothermal colorant incorporation.

The presence of labels, adhesives, or other materials attached to polystyrene products may affect the recycling process. In some cases, preprocessing steps such as sorting, washing, or grinding may be employed to prepare the polystyrene material for efficient recycling.

The molecular weight and degree of crosslinking of the polystyrene material may influence the recycling process. Higher molecular weight polystyrene or heavily crosslinked materials may require more energy or longer processing times to achieve complete depolymerization to styrene monomer.

In some cases, the polystyrene material may contain flame retardants, plasticizers, or other additives that may affect the recycling process or the purity of the recovered styrene monomer. The recycling method may need to account for these additives and may include additional purification steps if necessary.

The source and history of the polystyrene material may impact its suitability for recycling. Polystyrene that has been exposed to prolonged sunlight, high temperatures, or chemical environments may have undergone some degree of degradation or chemical changes, which may affect the recycling process and the quality of the recovered styrene monomer.

### Photothermal Colorant

The photothermal colorant may be a material capable of converting light energy into heat energy. In some cases, the photothermal colorant may be carbon black. Carbon black may be an effective photothermal colorant due to its ability to absorb light across a wide spectrum and efficiently convert that light into heat. In some cases, the photothermal colorant may be virgin photothermal colorant. In some cases, the photothermal colorant may be recovered photothermal colorant that was previously used in the recycling system.

Carbon black may be composed of fine particles of elemental carbon with a high surface area to volume ratio. This structure may allow carbon black to absorb light effectively and rapidly transfer the resulting heat to surrounding materials. The particle size of carbon black used as a photothermal colorant may vary, but in some cases may be in the nanometer to micrometer range.

In some cases, the photothermal colorant may be a material other than carbon black. Alternative photothermal colorants may include other carbon-based materials such as graphite, graphene, or carbon nanotubes. These materials may offer similar light absorption and heat generation properties to carbon black, while potentially providing different particle sizes or surface characteristics that may be advantageous for certain applications.

Metal-based nanoparticles may also be used as photothermal colorants in some cases. For example, gold, silver, or copper nanoparticles may exhibit strong photothermal effects due to their surface plasmon resonance properties. These metal nanoparticles may offer tunable absorption spectra based on their size and shape, potentially allowing for more targeted light absorption compared to carbon-based materials.

Organic dyes or pigments with strong light absorption properties may serve as photothermal colorants in some implementations. These materials may offer advantages in terms of color variety or ease of incorporation into certain polymer matrices.

The choice of photothermal colorant may depend on factors such as the specific wavelengths of light to be used, the desired heat generation efficiency, compatibility with the polystyrene matrix, and potential impacts on the recycled styrene monomer purity. In some cases, combinations of different photothermal colorants may be used to achieve desired properties or performance characteristics.

The concentration of the photothermal colorant within the polystyrene may vary depending on the specific colorant used and the desired heat generation efficiency. In some cases, the concentration may range from less than 1% to more than 10% by weight of the polystyrene. The optimal concentration may balance factors such as heat generation, light penetration depth, and potential impacts on the physical properties of the polystyrene or recycled styrene monomer. In some embodiments, the concentration may be no more than 1% by weight of the polystyrene. In some embodiments, the concentration may be 1%-3% by weight of the polystyrene. In some embodiments, the concentration may be 3%-5% by weight of the polystyrene. In some embodiments, the concentration may be 5%-8% by weight of the polystyrene. In some embodiments, the concentration may be 8%-10% by weight of the polystyrene. In some embodiments, the concentration may be 10%-20% by weight of the polystyrene.

The photothermal colorant may be incorporated into the polystyrene through various methods. In some cases, the colorant may be mixed with polystyrene pellets or powder before processing. Alternatively, the colorant may be added during the initial polymerization of styrene monomer to produce colored polystyrene. For recycling applications, the photothermal colorant may already be present in commercial polystyrene products, particularly in black or dark-colored items.

### Light Source

A light source 50 may be configured to generate light to irradiate the photothermal colorant (in this embodiment, the photothermal colorant within the reaction vessel 10).

The light source 50 may be selected from various types of illumination devices. In some embodiments, the light source 50 may be an LED (Light Emitting Diode), which offers advantages such as energy efficiency, long lifespan, and the ability to emit light at specific wavelengths that may be optimized for particular photothermal colorants. In various embodiments, the LED may be configured to emit one or more wavelengths of visible light (e.g., about 400 nm to about 700 nm wavelengths), ultraviolet light (e.g., about 10 nm to about 400 nm wavelengths), infrared light (e.g., about 700 nm to about 1 mm wavelengths), or a combination thereof. In some implementations, the LED may be adjustable to emit different wavelengths or intensities of light. In some implementations, the LED may be, e.g., a high intensity white LED. The white LED may emit light across a broad spectrum of visible wavelengths; such white LEDS typically range from approximately 400 nm to 700 nm, and often have peaks in the blue (e.g., 450 nm-480 nm) and yellow-green (e.g., 500 nm-590 nm, especially 560 nm-580 nm) regions of the spectrum.

In some embodiments, the light source 50 may be a laser, which can provide highly focused, monochromatic light that may efficiently activate certain photothermal colorants. In some embodiments, the light source 50 may be an incandescent bulb, which produces a broad spectrum of light and may be suitable for activating a wide range of photothermal colorants. In some embodiments, the light source 50 may be a halogen lamp, which provides intense white light that may be effective for generating heat in photothermal colorants. In some embodiments, the light source 50 may be a xenon lamp, which produces light similar to natural sunlight and may be particularly effective for simulating solar conditions in laboratory settings. In some embodiments, the light source 50 may be a fluorescent lamp, which offers energy efficiency and relatively low heat generation from the source itself. In some embodiments, the light source 50 may be a plasma lamp, which can provide high-intensity light across a broad spectrum. The specific type of light source 50 may be selected based on factors such as the absorption spectrum of the photothermal colorant, the desired rate of heat generation, and practical considerations such as cost and availability.

In some cases, the light source 50 may be sunlight. The recycling system 1 may be configured to utilize natural sunlight as the light source for irradiating the photothermal colorant. When using sunlight, the recycling system 1 may be positioned outdoors or near windows to allow direct exposure to sunlight.

The choice of light source 50 may affect various aspects of the recycling process. For example, using an LED as the light source 50 may allow for more precise control over the wavelength and intensity of light used to irradiate the photothermal colorant. This control may enable optimization of the heat generation process for different types of photothermal colorants or polystyrene materials.

When using sunlight as the light source 50, the recycling process may be more dependent on environmental factors such as time of day, weather conditions, and geographical location. However, utilizing sunlight may provide a more sustainable and energy-efficient approach to the recycling process.

In some cases, the recycling system 1 may be designed to accommodate multiple types of light sources 50. For example, the system may be configured to use artificial light sources such as LEDs during periods of low sunlight availability, and switch to using sunlight when conditions are favorable. This flexibility may allow for continuous operation of the recycling process regardless of external lighting conditions.

The intensity and duration of light exposure from the light source 50 may be adjusted based on factors such as the type and concentration of photothermal colorant, the volume and form of polystyrene being processed, and the desired rate of styrene monomer production. In some implementations, the recycling system 1 may include sensors and control mechanisms to automatically adjust the light source 50 parameters for optimal performance.

When irradiated with light, the photothermal colorant may absorb photons and convert their energy into heat through various mechanisms such as non-radiative relaxation or phonon generation. This localized heat generation may then transfer to the surrounding polystyrene molecules, providing the thermal energy necessary to break chemical bonds and initiate the depolymerization process. This may begin to generate one or more smaller molecules, and preferably, generate styrene monomer.

### Condenser

The recycling system 1 may optionally (and preferably) include a condenser 20 operably coupled to the reaction vessel 10. The condenser 20 may be designed to cool and condense vapors produced during the polystyrene degradation process. In some cases, the condenser 20 may include ports for an incoming coolant 22 and an outgoing coolant 24. The coolant may be a fluid, such as a liquid, circulating through the condenser 20 to facilitate the condensation of styrene monomer vapor.

In some implementations, the coolant may be water, which may provide efficient heat transfer and be environmentally friendly. The temperature of the incoming coolant 22 may be controlled to optimize the condensation process. As the coolant flows through the condenser 20, the coolant may absorb heat from the vapor, causing the vapor to condense into liquid styrene monomer. The outgoing coolant 24 may then exit the condenser 20 at a higher temperature.

The condenser 20 may be designed with features to enhance the condensation process. In some cases, the condenser 20 may have an internal structure with a large surface area, such as a series of coils or plates, to maximize contact between the vapor and the cooled surfaces. This design may improve the efficiency of the condensation process.

### Receiving Vessel

A receiving vessel 30 may be configured to receive and collect molecules (and preferably, monomers such as styrene monomers) from the reaction vessel. In some embodiments, a condenser 20 is disposed between the reaction vessel 10 and the receiving vessel 30.

The various vessels and components may be connected in any appropriate manner, including, e.g., direct connection, as well as indirect connection via, e.g, tubing or piping.

The receiving vessel 30 may be configured to receive and store the liquid styrene monomer as the monomer drips or flows from the condenser 20. In some implementations, the receiving vessel 30 may be made of a material that is resistant to chemical interaction with styrene monomer, such as glass or certain types of plastic.

The receiving vessel 30 may be designed with features to facilitate the collection and storage of the styrene monomer. In some cases, the receiving vessel 30 may have a wide mouth to ensure efficient collection of the condensed monomer. The receiving vessel 30 may also include a sealed or sealable top to prevent contamination or loss of the collected styrene monomer.

In some implementations, the receiving vessel 30 may be equipped with a cooling system to maintain the collected styrene monomer at a low temperature. This cooling may help prevent re-polymerization or other unwanted reactions of the styrene monomer.

The arrangement of the condenser 20 and receiving vessel 30 may allow for the efficient distillation of styrene monomer from the reaction vessel 10. As the polystyrene in the reaction vessel 10 degrades due to the heat generated by the photothermal colorant, styrene monomer vapor may rise and enter the condenser 20. The vapor may then condense in the condenser 20 and flow as a liquid into the receiving vessel 30.

In some cases, the receiving vessel 30 may be removable from the recycling system 1. This feature may allow for easy collection and transfer of the styrene monomer for further processing or use. The receiving vessel 30 may also include markings or a transparent section to allow for visual monitoring of the collected styrene monomer volume. In some cases, the receiving vessel may have an outlet for removing the collected styrene monomer.

### Vacuum Pump

The recycling system 1 may include a vacuum system to facilitate the degradation of polystyrene and the collection of styrene monomer. As shown in FIG. 1, the vacuum system may comprise a vacuum pump 40 operably coupled to the receiving vessel 30. The vacuum pump 40 may be configured to create and maintain a reduced pressure environment within the recycling system 1.

In some cases, the vacuum pump 40 may be a mechanical pump, such as a rotary vane pump or a diaphragm pump. In other cases, the vacuum pump 40 may be an oil-free pump, which may help prevent contamination of the styrene monomer. The vacuum pump 40 may be sized appropriately to achieve and maintain the desired level of vacuum within the recycling system 1.

A pressure gauge 42 may be operably coupled to the receiving vessel 30 to monitor the pressure within the recycling system 1. The pressure gauge 42 may provide real-time pressure readings, allowing for precise control of the vacuum conditions. In some implementations, the pressure gauge 42 may be a digital pressure sensor capable of interfacing with a control system to automatically adjust the vacuum pump 40 operation based on the measured pressure.

The vacuum system may play a crucial role in the recycling process by creating a dynamic vacuum environment. In some cases, the styrene monomer may be formed in the reaction vessel 10 under a dynamic vacuum. The dynamic vacuum may help to continuously remove the styrene monomer as the polystyrene degrades, shifting the equilibrium of the depolymerization reaction towards the formation of more styrene monomer.

The reduced pressure created by the vacuum system may lower the boiling point of the styrene monomer, facilitating its vaporization and subsequent condensation in the condenser 20. This process may allow for the efficient separation of the styrene monomer from any remaining polystyrene or other byproducts of the degradation process.

In some implementations, the vacuum system may be designed to maintain a specific pressure range that optimizes the depolymerization and distillation processes. The optimal pressure range may depend on factors such as the temperature in the reaction vessel 10, the type of polystyrene being recycled, and the desired rate of styrene monomer production.

The vacuum system may also help to remove any air or other gases present in the recycling system 1, which may interfere with the depolymerization process or contaminate the final styrene monomer product. By maintaining a low-oxygen environment, the vacuum system may help prevent unwanted oxidation reactions during the recycling process.

In some cases, the vacuum system may be equipped with traps or filters to prevent any liquid or solid particles from entering and potentially damaging the vacuum pump 40. These traps may also serve to collect any volatile byproducts of the depolymerization process that may not condense in the condenser 20.

The operation of the vacuum system may be coordinated with other components of the recycling system 1, such as the light source 50 and the condenser 20, to optimize the overall efficiency of the styrene monomer production process. For example, the vacuum level may be adjusted based on the temperature readings from the thermometer 12 to maintain optimal conditions for polystyrene degradation and styrene monomer collection.

### Light Directing Component

Optionally, a light directing component 52 may be disposed between the light source and any photothermal colorant to guide or focus the light onto the photothermal colorant. The light directing component may include one or more fiber optic cables. The light directing component may include one or more lenses, such as a fresnel lens. The Fresnel lens may be used to concentrate sunlight onto the photothermal colorant, potentially increasing the efficiency of the heat generation process.

### Temperature Sensors

Referring to FIG. 1, a thermometer 12 or other temperature sensor may be operably coupled to the reaction vessel 10, allowing for temperature monitoring during the recycling process. The thermometer 12 may be, e.g., a digital thermometer, a thermocouple, or another temperature sensing device capable of accurately measuring the elevated temperatures involved in the polystyrene degradation process.

### Pressure Sensors

In some embodiments, a pressure gauge 42 or other pressure sensor may be operably coupled to the receiving vessel 30, allowing for monitoring of the system's internal pressure.

### Control Systems

In some embodiments, the control system may be operably coupled to one or more components, such as a temperature sensor, a pressure gauge, and/or a flow meter to monitor and control the recycling process. The control system 54 may be configured to adjust the light source, vacuum source, or other parameters based on the monitored conditions. For example, in some cases, the thermometer 12 may be connected to a control system 54 that may be configured to adjust the light source 50 or other parameters (such as a positioning or orientation of the light directing component 52) based on the measured temperature.

This arrangement of components may allow for the controlled photothermal depolymerization of polystyrene, with the ability to monitor and adjust key parameters such as temperature, pressure, and light exposure throughout the recycling process.

The efficiency of heat generation and transfer from the photothermal colorant to the polystyrene may depend on factors such as the thermal conductivity of the materials, the dispersion of the colorant within the polymer matrix, and the intensity and wavelength of the incident light. Optimizing these factors may lead to more efficient and effective polystyrene recycling processes.

The recycling system may be designed to accommodate different scales of operation, from small laboratory setups to larger industrial-scale systems. The size and capacity of the vessels, light source, and vacuum source may be scaled accordingly to meet the desired throughput of polystyrene recycling.

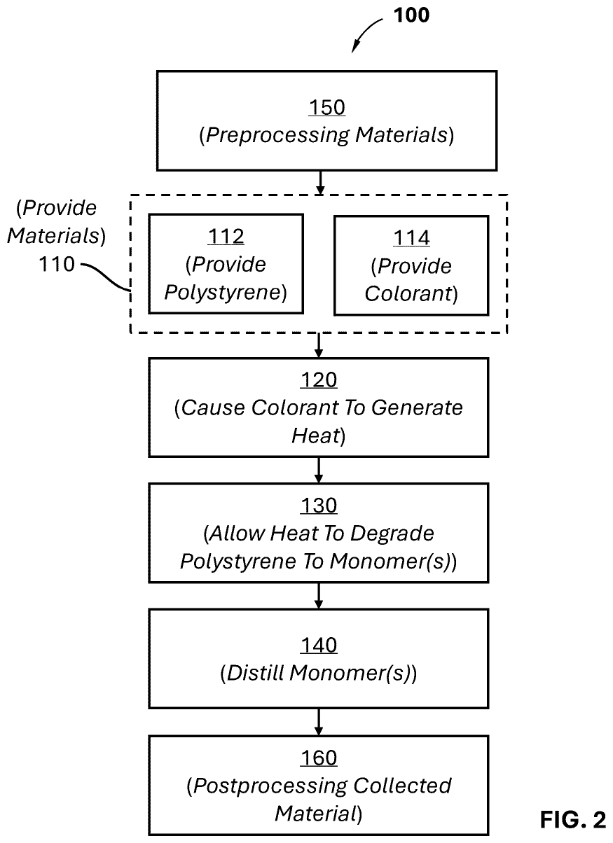

The recycling system 1 may implement a method 100 for recycling polystyrene, as illustrated in FIG. 2. The method 100 may include several steps designed to efficiently convert polystyrene into styrene monomer using photothermal conversion.

The method 100 may begin with a step of providing materials 110. This step may optionally involve two sub-steps: a step of providing polystyrene 112 and a step of providing photothermal colorant 114. In some cases, the polystyrene and photothermal colorant may be provided separately and combined in the reaction vessel 10. In other cases, the polystyrene may already contain the photothermal colorant, such as in black polystyrene products.

Following the provision of materials, the method 100 may proceed to a step of causing photothermal colorant to generate heat 120. This step may involve irradiating the photothermal colorant with light from the light source 50. The light directing component 52 may be used to focus or concentrate the light onto the photothermal colorant within the reaction vessel 10.

In some cases, the step of causing photothermal colorant to generate heat 120 may occur for a period of time of at least 5 minutes, at least 10 minutes, or at least 15 minutes. In some embodiments, the period of time may be no more than 10 minutes, no more than 15 minutes, no more than 20 minutes, no more than 30 minutes, or no more than 60 minutes. In preferred embodiments, the period of time may be 15 minutes-60 minutes. In some embodiments, the period of time may be 1 hour-8 hours. In some embodiments, the period of time may be at least 8 hours.

This duration may allow sufficient time for the photothermal colorant to absorb light energy and convert it into heat. The thermometer 12 may be used to monitor the temperature within the reaction vessel 10 during this step.

The period of time for irradiating the photothermal colorant may vary depending on factors such as the type and concentration of photothermal colorant, the intensity of the light source 50, and the volume of polystyrene being processed. In some cases, the period of time may be 15-60 minutes. This range may provide a balance between efficient heat generation and practical processing times for many applications.

In some implementations, the period of time for irradiating the photothermal colorant may be at least 60 minutes. Extended irradiation times may be used for larger volumes of polystyrene or when using lower intensity light sources. The recycling system 1 may be designed to accommodate these longer processing times while maintaining stable conditions within the reaction vessel 10.

The method 100 may then proceed to a step of allowing heat to degrade polystyrene 130. During this step, the heat generated by the photothermal colorant may cause the polystyrene to break down into smaller molecules, primarily styrene monomer. The temperature within the reaction vessel 10 may be maintained at or above the depolymerization temperature of polystyrene, which may be about 395 °C. As is known, the exact depolymerization temperature of a polystyrene can vary depending on several factors, including, e.g., (i) the molecular weight of the polystyrene, (ii) the presence of additives or fillers, (iii) the heating rate, (iv) the atmosphere (inert vs. oxidative), and (v) the specific grade of polystyrene (general purpose, high impact, etc.). In some embodiments, the depolymerization temperature of polystyrene may be approximately 350-400 °C (662-752 °F). At these temperatures, polystyrene begins to break down into its constituent monomer (styrene) and other decomposition products.

The amount of light energy directed towards the photothermal colorant may be controlled to prevent the temperature from being too high. For example, if polystyrene is heated to temperatures significantly above its depolymerization range (e.g., 450 °C), several undesirable effects may occur, including, e.g., (i) complete thermal degradation (rather than clean depolymerization to styrene monomers, the polymer chains break down randomly, producing a complex mixture of styrene monomers (in reduced yield), benzene, toluene, and ethylbenzene, various other aromatic compounds, and carbon-rich residue/char), (ii) formation of toxic byproducts (at very high temperatures, such as above 500 °C, more hazardous compounds can form, including polycyclic aromatic hydrocarbons (PAHs), dioxins (if chlorinated additives are present), carbon monoxide, and volatile organic compounds (VOCs)), and/or (iii) combustion (if oxygen is present, the polystyrene and its breakdown products will combust rather than just depolymerize).

Finally, the method 100 may include a step 140 of distilling the breakdown products, including (preferably) a monomer such as (most preferably) a styrene monomer. In this step, the styrene monomer produced from the degradation of polystyrene may be separated and collected. The distillation process may take advantage of the reduced pressure environment created by a vacuum source connected to the reaction vessel 10.

The efficiency of the recycling method 100 may be influenced by various factors. For example, the ratio of photothermal colorant to polystyrene may affect the rate of heat generation and, consequently, the rate of polystyrene degradation. The particle size and dispersion of the photothermal colorant within the polystyrene matrix may also impact the efficiency of heat transfer and degradation.

In some cases, the method 100 may be modified to include additional steps or variations. For instance, one or more preprocessing steps 150 may be added to, e.g., prepare the polystyrene material, such as by grinding or sorting. One or more post-processing steps 160 may also be included to, e.g., purify the collected styrene monomer or to recover and reuse the photothermal colorant.

The recycling method 100 may be adapted for continuous or batch processing depending on the specific requirements of the recycling operation. In a continuous process, fresh polystyrene material may be continuously fed into the reaction vessel 10 while styrene monomer is continuously distilled and collected. In a batch process, discrete quantities of polystyrene may be processed in cycles.

The method 100 may be optimized for different types of polystyrene materials, including virgin polystyrene, post-consumer waste, or industrial scrap. The specific parameters of each step, such as irradiation time, temperature, and pressure, may be adjusted to accommodate the characteristics of the input material and to maximize the yield of styrene monomer.

### EXAMPLES

Disclosed is a unique strategy to mitigate black plastic pollution and facilitate a circular economy. By taking advantage of existing photothermally active additives in black plastics, we use light to depolymerize commercial plastics back to monomers (FIG. 1D). Using mild, visible light irradiation of carbon black enabled polymerization and subsequent depolymerization of black polystyrene (PS), including styrene copolymers. Notably, the disclosed method also proved effective for depolymerizing post-consumer waste black plastics. Photothermal depolymerization using existing plastic additives presents a new pathway for chemical recycling, allowing mild reaction conditions and lower overall energy input.

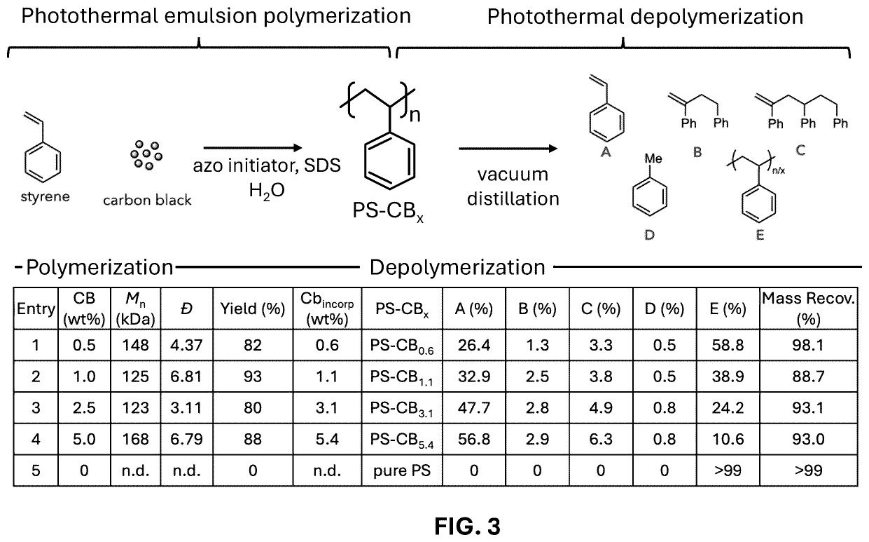

The feasibility of carbon black-mediated photothermal depolymerization was first tested in a controlled system. A commercially relevant emulsion polymerization method was utilized to prepare PS embedded with carbon black to quickly access high molecular weight polymer, using water as a “green” solvent. Because carbon black is an efficient photothermal agent, it was hypothesized that low-intensity 660 nm LEDs that exhibit greater penetration depth could thermally activate the radical initiator for polymerization. Near quantitative styrene conversion to high molecular weight PS was observed in the presence of 0.5 to 5 wt % of carbon black (see FIG. 3, entries 1-4). However, no polymerization occurred without carbon black, showing that photothermal conversion was necessary to activate the initiator (see FIG. 3, entry 5).

By filtering the polymer precipitates, we obtained >80 % yield of PS-CB material in all cases. Carbon black was recovered quantitatively with the polymer particles, providing the isolated polymer material ranging from 0.6 to 5.4 wt % carbon black (denoted PS-CBx where x is the weight percent). After synthesizing the PS-CB samples, we proceeded to investigate photothermal depolymerization studies. To achieve efficient depolymerization, local temperatures of carbon black should be above the Tc, the temperature at which the polymerization and depolymerization rates are equal. To exceed 395 °C (Tc for styrene), a high-intensity white LED light was chosen to maximize heat transfer from carbon black to PS.

The PS-CB samples with different carbon black loadings were screened using photothermal depolymerization under vacuum distillation for 30 min (see FIG. 3). The primary product from depolymerization was styrene monomer, with dimer, trimer, and toluene also detected. Increasing amounts of styrene monomer with increasing carbon black wt % was observed, reaching a maximum of 57% styrene monomer with high mass recovery. Other byproducts were detected in similar ratios in each sample, regardless of carbon black loading (see FIG. 3, entries 1-4). Pure PS did not undergo depolymerization without carbon black under white LED irradiation, indicating that photothermal conversion facilitated the process (see FIG. 3, entry 5).

Analytically pure styrene, dimer, and trimer were successfully separated in the receiving flask, the condenser, and the reaction vial, respectively. These samples were assessed with gas chromatography-mass spectrometry (GC-MS) to confirm the formation of all the products. The loss of mass recovery was accounted for to a small amount of styrene escaping the receiving flask and a minute amount of other byproducts, such as α-methylstyrene and 1,3-diphenylpropane. Despite these, after photothermal depolymerization, 1H NMR and GC-MS spectra showed a clean product distribution of monomer, dimer, and trimer.

To highlight the reusability of all system components, a recycling sequence of polymerization followed by depolymerization was executed. First, the contents from the recovery flask were passed through basic alumina and resynthesized PSᵣ-CB_{6.0} via photothermal emulsion polymerization. The used carbon black was separated from the reaction vial mixture through column chromatography and it was reused for the photothermal emulsion polymerization with commercial styrene (PS-CBr_{7.2}). Both samples were then depolymerized and provided nearly identical results (57-58%) to the original samples. Lastly, to show that the mass balance of PS-CB after depolymerization could be repurposed, the leftover PS-CB_{5.5} was diluted with styrene monomer. An aliquot from the stock solution was used directly in photothermal emulsion polymerization without extra carbon black (to produce PSr-CBr_{0.6}) or additional carbon black to make PSr-CBr5.6. Both samples were depolymerized, delivering 57 % styrene in the case of PSr-CBr_{5.6}. These results showed that every component recovered from the system can be repurposed to produce new PS-CB, which can be depolymerized with comparable efficiencies, highlighting the circularity of the disclosed process.

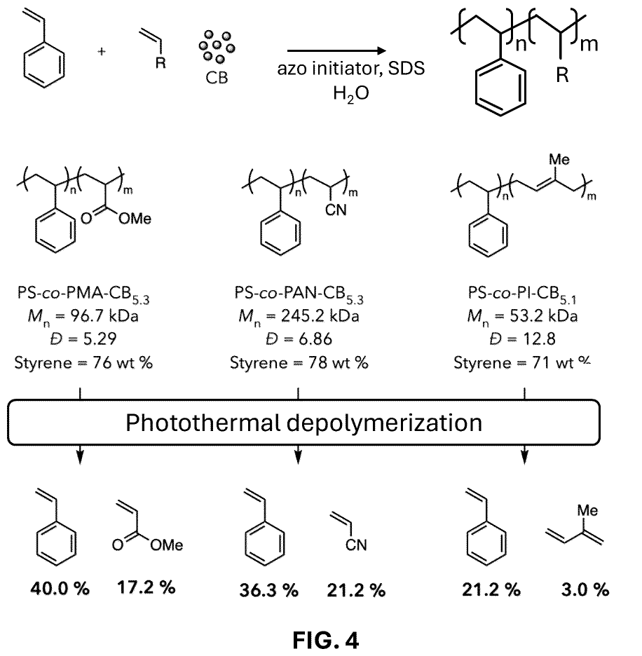

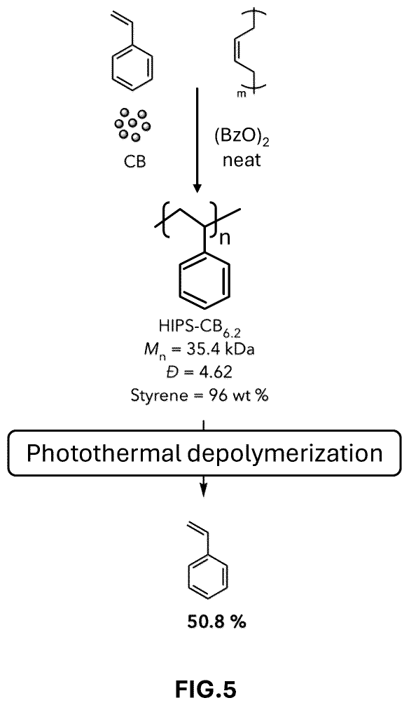

Styrene copolymers are of interest in depolymerization, as styrene is frequently used in copolymer synthesis to create new materials. It was hypothesized that the disclosed system would be amenable to copolymer depolymerization. Styrene was first copolymerized with methyl acrylate (PS-co-PMA-CB_{5.3}), acrylonitrile (PS-co-PAN-CB_{5.3}), and isoprene (PS-co-PI-CB_{5.1}) via photothermal emulsion syntheses (see FIG. 4). High impact polystyrene (HIPS-CB6.2) was also synthesized through graft copolymerization of styrene on polybutadiene (PB) using photothermal bulk polymerization (see FIG. 5). These copolymers were then subjected to photothermal depolymerization for one hour using the distillation protocol (see FIGS. 4-5). A moderate amount of styrene and small amounts of comonomers, like methyl acrylate, acrylonitrile, and isoprene, were detected. It was expected that copolymer depolymerization would be more challenging for the following reasons. Since these comonomers have higher ceiling temperatures, the copolymers are less likely to depropagate than pure polystyrene. Moreover, these copolymers would be more prone to undergo side reactions, especially for PS-co-PI-CB_{5.1}, owing to the double bonds on the backbone. Another PS-co-PI-CB_{5.2} was synthesized (with 30 wt % styrene) and a 14% styrene yield was observed after photothermal depolymerization. This result was even lower than the 21% styrene yield in the case of PS-co-PI-CB_{5.1} (with 71 wt % styrene), possibly because the higher double bond concentration caused more side reactions to prevent styrene formation. Despite these challenges, the overall results showed a potential for using photothermal depolymerization as a promising avenue for deconstructing styrene copolymers.

The disclosed system was tested on commercial polystyrene samples to demonstrate the applicability of the photothermal depolymerization method and its potential industrial value on post-consumer waste plastic recycling. Post-consumer black polystyrene samples, including foamed polystyrene (PS foam), food containers, coffee cup lids, etc., were subjected to our standard depolymerization conditions without additional carbon black or other metal catalysts. Excitingly, eight black polystyrene plastics were successfully depolymerized to styrene monomer in up to 53% yield. Specifically, a PS Foam Tray (53.4±0.4%), a film container (40.8±1.5%), a sushi box (36.2±4.5%), a flower pot (28.8±3.9%), an LED container (40.4±4.1%), a coffee cup lid (35.0±2.2%), a cake tray (38.0±1.6%), and 3D printer JIPS feed material (36.9±2.0%). Despite these materials containing variable amounts of carbon black or other additives, styrene yields >30 % were obtained, highlighting the generality of the approach and tolerance to variation.

In addition, white or clear polystyrene samples were melt processed with carbon black to achieve 5 wt % carbon black films. Under the same depolymerization conditions as above, similar styrene yields were observed (up to 54%), comparable to the photothermal depolymerization of the as-synthesized PS-CB mixture. Specifically, a PS foam tray (52.3±1.2%), a coffee cup lid (28.6±0.3%), a container lid (50.8±1.3%), and a clear cup (53.9±0.9%).

Furthermore, depolymerization was performed on scales of 3 and 6 g of this material and achieved styrene yields in up to 44%. These results indicate that carbon black could be added post-use to non-black plastic samples and still perform efficient depolymerization.

Post-consumer PS wastes often come into contact with food or other contaminants (56). To mimic these conditions, photothermal depolymerization of black PS foam was conducted in the presence of various food contaminants (20 wt %) to test the robustness of the protocol. Gratifyingly, the monomer recovery of the sample with sucrose remained almost identical to the uncontaminated samples (53%). Canola oil and soy sauce slightly lowered the styrene yield to 46 %. Additionally, despite having a radical scavenger (vitamin C or ascorbic acid) that could quench radicals on the polymer backbone, the styrene recovery was still 37 % in the presence of orange juice.

Lastly, using a Fresnel lens, we irradiated commercial PS with focused sunlight under static vacuum. The black PS foam sample yielded 70% styrene after 15 minutes, while the pure white commercial PS powder stayed intact (0% styrene) as a control group. The higher reaction efficiency was attributed to the greater light intensity achieved by the focal point of the lens, as compared to our LEDs. These results indicated that the method needs no energy input beyond sunlight and has significant potential for real-world application as an efficient and highly tolerant method for closed-loop recycling.

A number of implementations have been described. Nevertheless, it will be understood that various modifications may be made without departing from the spirit and scope of the disclosure. Accordingly, other implementations are within the scope of the following claims.

What is claimed is:

1. A method for recycling polystyrene, comprising:
providing a photothermal colorant and a polystyrene, the photothermal colorant being operably coupled to the polystyrene;
causing the photothermal colorant to generate heat by irradiating the photothermal colorant with light; and
forming a styrene monomer by allowing the heat to degrade the polystyrene.

2. The method of claim 1, wherein the photothermal colorant is carbon black.

3. The method of claim 1, wherein the photothermal colorant is a material other than carbon black.

4. The method of claim 1, wherein the photothermal colorant and the polystyrene are separately added to a vessel.

5. The method of claim 1, wherein the polystyrene is a polystyrene copolymer.

6. The method of claim 5, wherein the polystyrene copolymer is selected from the group consisting of a styrene-acrylic copolymer, a styrene-butadiene copolymer, a styrene-acrylonitrile copolymer, a styrene-acrylonitrile-butadiene copolymer, and a styrene-isoprene copolymer.

7. The method of claim 1, wherein the photothermal colorant and the polystyrene are disposed within one or more products configured to be recycled.

8. The method of claim 1, wherein the styrene monomer is formed in a vessel under a dynamic vacuum.

9. The method of claim 8, wherein the styrene monomer is distilled to a receiving vessel.

10. The method of claim 1, wherein the photothermal colorant generates sufficient heat to expose the polystyrene to a temperature of at least about 395 °C.

11. The method of claim 1, wherein irradiating the photothermal colorant with light comprises exposing the photothermal colorant to radiation emitted from an LED.

12. The method of claim 1, wherein irradiating the photothermal colorant with light comprises exposing the photothermal colorant to radiation emitted from the sun.

13. The method of claim 12, further comprising directing sunlight to the photothermal colorant using a Fresnel lens.

14. The method of claim 1, wherein irradiating the photothermal colorant with light occurs for a period of time of at least 5 minutes.

15. The method of claim 14, wherein the period of time is 15-60 minutes.

16. The method of claim 14, wherein the period of time is at least 60 minutes.

17. A system for polystyrene recycling, comprising:
a first vessel configured to receive a photothermal colorant and a polystyrene;
a light source configured to irradiate the photothermal colorant within the first vessel;
a vacuum source configured to be operably coupled to the first vessel; and
a second vessel configured to receive a styrene monomer from the first vessel.

18. The system of claim 17, further comprising a condenser operably coupled to the first vessel and second vessel, and disposed between the first vessel and the second vessel.
